SEQUENCE LISTING (14 sequences)

SEQ ID NO: 1 - protein, 15 aa, Artificial
  Synthetic
ADGSSWEGVGVVPDV

SEQ ID NO: 2 - protein, 23 aa, Artificial
  Synthetic
PTARSVGAADGSSWEGVGVVPDV

SEQ ID NO: 3 - protein, 23 aa, Artificial
  Synthetic
HVDDTDLYLTIPTARSVGAADGS

SEQ ID NO: 4 - protein, 8 aa, Artificial
  Synthetic
TSSEVATE

SEQ ID NO: 5 - protein, 8 aa, Artificial
  Synthetic
TSSEAATE

SEQ ID NO: 6 - protein, 8 aa, Artificial
  Synthetic
DTNLASST

SEQ ID NO: 7 - protein, 8 aa, Artificial
  Synthetic
DTALASST

SEQ ID NO: 8 - protein, 18 aa, Artificial
  Synthetic
DTNLASSTIIKEGIDKTV

SEQ ID NO: 9 - protein, 22 aa, Artificial
  Synthetic
VPLLANNRERRGIAKDGKIKHE

SEQ ID NO: 10 - protein, 20 aa, Artificial
  Synthetic
TSSEVATEVPFRLMHPQPED

SEQ ID NO: 11 - protein, 20 aa, Artificial
  Synthetic
SLTKTLTLVPLLANNRERRG

SEQ ID NO: 12 - protein, 20 aa, Artificial
  Synthetic
SLTRTLTLLPLLANNRERAG

SEQ ID NO: 13 - protein, 21 aa, Artificial
  Synthetic
KEGIDKTVMGILVSYQIKVKL

SEQ ID NO: 14 - protein, 21 aa, Artificial
  Synthetic
KEGIDRTVLGILVSYQIKVKL

(19) United States
(12) Patent Application Publication
(10) Pub. No.: US 2005/0220803 A1
(43) Pub. Date: Oct. 6, 2005

(54) Use of an organ-specific self-pathogen for treatment of a non-autoimmune disease of said organ

(75) Inventors: Michal Eisenbach-Schwartz, Rehovot (IL); Tal Mizrahi, Moshav Ge'alya (IL)

Correspondence Address: BROWDY AND NEIMARK, P.L.L.C.;624 NINTH STREET, NW, SUITE 300, WASHINGTON, DC, 20001-5303, US

(73) Assignee: Yeda Research and Development Co. Ltd., Rehovot (IL)

(21) Appl. No.: 10/509,180

(22) PCT Filed: Mar. 25, 2003
(86) PCT No.: PCT/IL03/00251

§ 371 Date: Apr. 27, 2005

Related U.S. Application Data
(60) Provisional application No. 60/367,271, filed on Mar. 26, 2002.

(51) Int. Cl. A61K 39/00; C07K 14/705

(52) U.S. Cl. 424/185.1; 530/350

(57) ABSTRACT

An agent selected from: (a) a pathogenic self-antigen associated with a T-cell-mediated specific autoimmune disease of an organ; (b) a peptide which sequence is comprised within the sequence of (a); (c) a peptide obtained by modification of (b), by replacement of one or more amino acid residues by different amino acid residues, said modified peptide still being capable of recognizing the T-cell receptor recognized by the parent peptide but with less affinity; (d) a nucleotide sequence encoding (a), (b), or (c); and (e) T cells activated by a pathogenic self-antigen of (a), a peptide of (b), or a modified peptide of (c), can be used for treatment of a non-autoimmune disease, disorder or injury in said organ. For example, uveitogenic antigens or peptides thereof can be used for treatment of a non-autoimmune disease, disorder or injury in the eye.

### FIELD OF THE INVENTION

The present invention is in the field of Immunology and relates to pathogenic self-antigens associated with a T-cell-mediated specific autoimmune disease in an organ, or fragments thereof, and their use for treating a non-autoimmune disease, disorder or injury in said organ. In a particular example, the organ is the eye and an uveitogenic antigen, a peptide thereof or an analog of said peptide are applied for the treatment of a non-autoimmune disease, disorder or injury in the eye.

Abbreviations: CFA: complete Freund's adjuvant; CNS: central nervous system; 4-Di-10-Asp: 4-(4(didecylamino)styryl)-N-methylpyridinium iodide; EAE: experimental autoimmune encephalomyelitis; EAU: experimental autoimmune uveoretinitis; IFA: incomplete Freund's adjuvant; IRBP: interphotoreceptor retinoid-binding protein; MBP: myelin basic protein: PBS: phosphate-buffered saline; RGC: retinal ganglion cell; S—Ag: soluble antigen.

### FIELD AND BACKGROUND OF THE INVENTION

Axonal injury in the central nervous system (CNS) leads to an inevitable process of degeneration, not only in the afflicted axons but also in neighboring axons that escaped the initial insult (Yoles and Schwartz, 1998a). This secondary degeneration has been attributed to self-destructive compounds that emerge from the degenerating axons into the micro-environment at the lesion site, making it hostile to the remaining tissue.

We recently discovered that CNS myelinated axons, after suffering a mechanical insult such as a crush injury, can benefit from the activity of autoreactive T cells directed against myelin antigens (Hauben et al, 2000a,b; Moalem et al, 1999, 2000). We further found that the neuroprotective activity exhibited by these autoimmune T cells is not merely the result of an experimental manipulation, but is a physiological way in which the body copes with stressful conditions (Schori et al, 2001a; Yoles et al, 2001). Accordingly, we proposed that just as the immune system is called upon to defend the body from invading microbes, it is also needed to protect it from self-compounds that under conditions of trauma or stress (not necessarily related to pathogens) become toxic.

Interestingly, in the case of damage to myelinated CNS axons, the T cells that induce neuroprotection have the same specificity and phenotype as those known to cause autoimmune disease. Thus, the cells are both potentially protective and potentially destructive, and their actual expression evidently depends on how they are regulated. This might explain the observed correlation between the ability to manifest an autoimmune response with a beneficial outcome and the ability to resist the development of an autoimmune disease (Kipnis et al, 2001). Therefore, the ability to protect neuronal tissue apparently does not correspond to a lack of autoimmunity, but, rather, reflects autoimmunity that is well controlled.

We were interested in investigating whether the T cells recruited in the specific environments of different injury sites for the purpose of coping with the local stressful situation have the same or different antigenic specificities. Our previous work indicated that although passive transfer of anti-myelin autoimmune T cells (Moalem et al, 1999) or vaccination with myelin antigens (Kipnis et al, 2002) can protect retinal ganglion cells (RGCs) (Yoles and Schwartz, 1998a) after an insult to the optic nerve axons, these procedures are not protective after a direct insult to the RGCs themselves (Schori et al, 2001b). This finding led us to consider the possibility that each tissue has its own specific self-antigens that signal the immune system when the tissue needs help. In the case of axotomy, since the antigens that send signals summoning the immune system to the aid of the stressed neurons are myelin proteins associated not with neurons but with oligodendrocytes, we considered the possibility that the relevant antigens are not necessarily expressed on the cells that require assistance but on other cells in the vicinity. In addition, if an autoimmune disease is indeed the outcome of failure to control an autoimmune response whose original purpose was beneficial, it seems reasonable to postulate that the protection (beneficial response) and the disease (destructive response) share the same antigenic specificity.

In recent years, peptides derived from a pathogenic self-antigen associated with an autoimmune disease or analogs thereof have been proposed for treatment of the autoimmune disease. For example, peptide analogs of human myelin basic protein (MBP) have been described for treatment of multiple sclerosis (U.S. Pat. No. 5,948,764; U.S. Pat. No. 6,329,499); peptide analogs of the 65 kD isoform of human glutamic acid decarboxylase (GAD) and of insulin have been proposed for treatment of diabetes (U.S. Pat. No. 5,945,401 and U.S. Pat. No. 6,197,926, respectively); and an autoantigen derived from the retina such as S-antigen (S—Ag) and interphotoreceptor retinoid-binding protein (IRBP), or fragments thereof, have been described for the treatment of autoimmune uveoretinitis (U.S. Pat. No. 5,961,977).

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### SUMMARY OF THE INVENTION

It has now been found, in accordance with the present invention, that a tissue-specific self-antigen that is associated with an autoimmune disease in an organ, or a fragment of said self-antigen, can confer protective immunity to a non-autoimmune injury, disease, or disorder of said organ. This is contrary to what has been described in the prior art as mentioned above in which the pathogenic self-antigens associated with an autoimmune disease, and fragments thereof, have been disclosed for the treatment of the autoimmune disease itself.

The present invention relates, in one aspect, to a method for treating a disease, disorder or injury in an organ which is susceptible to a T-cell-mediated specific autoimmune disease, wherein said organ disease, disorder or injury is other than an autoimmune disease, the method comprising immunizing an individual having such a disease, disorder or injury with an agent selected from the group consisting of:
(a) a pathogenic self-antigen associated with a T-cell-mediated specific autoimmune disease of said organ;
(b) a peptide which sequence is comprised within the sequence of said pathogenic self-antigen of (a);
(c) a peptide obtained by modification of the peptide of (b), which modification consists in the replacement of one or more amino acid residues of the peptide by different amino acid residues, said modified peptide still being capable of recognizing the T-cell receptor recognized by the parent peptide but with less affinity (hereinafter “modified peptide”);
(d) a nucleotide sequence encoding a pathogenic self-antigen of (a), a peptide of (b) or a modified peptide of (c); and
(e) T cells activated by a pathogenic self-antigen of (a), a peptide of (b) or a modified peptide of (c).

In another aspect, the present invention relates to a pharmaceutical composition for treating a disease, disorder or injury in an organ which is susceptible to a T-cell-mediated specific autoimmune disease, wherein said organ disease, disorder or injury is other than an autoimmune disease, the composition comprising an agent selected from the group consisting of:
(a) a pathogenic self-antigen associated with a T-cell-mediated specific autoimmune disease of said organ;
(b) a peptide which sequence is comprised within the sequence of said pathogenic self-antigen of (a);
(c) a peptide obtained by modification of the peptide of (b), which modification consists in the replacement of one or more amino acid residues of the peptide by different amino acid residues, said modified peptide still being capable of recognizing the T-cell receptor recognized by the parent peptide but with less affinity (hereinafter “modified peptide”);
(d) a nucleotide sequence encoding a pathogenic self-antigen of (a), a peptide of (b) or a modified peptide of (c); and
(e) T cells activated by a pathogenic self-antigen of (a), a peptide of (b) or a modified peptide of (c).

In still a further aspect, the present invention relates to the use of an agent selected from the group consisting of:
(a) a pathogenic self-antigen associated with a T-cell-mediated specific autoimmune disease of said organ;
(b) a peptide which sequence is comprised within the sequence of said pathogenic self-antigen of (a);
(c) a peptide obtained by modification of the peptide of (b), which modification consists in the replacement of one or more amino acid residues of the peptide by different amino acid residues, said modified peptide still being capable of recognizing the T-cell receptor recognized by the parent peptide but with less affinity (hereinafter “modified peptide”);
(d) a nucleotide sequence encoding a pathogenic self-antigen of (a), a peptide of (b) or a modified peptide of (c); and
(e) T cells activated by a pathogenic self-antigen of (a), a peptide of (b) or a modified peptide of (c),
for the manufacture of a pharmaceutical composition for treatment of a disease, disorder or injury of said organ, excluding an autoimmune disease or disorder of the organ.

In a preferred embodiment, the organ is the eye and the antigen is a pathogenic ocular self-antigen associated with a T-cell-mediated specific autoimmune disease of the eye such as, but not limited to, an uveitogenic antigen selected from interphotoreceptor retinoid-binding protein (hereinafter “IRBP”), the S-antigen (S—Ag); and rhodopsin. These antigens or fragments thereof are useful for treatment of a non-autoimmune disease, injury or disorder of the eye.

### DESCRIPTION OF THE FIGURES

FIG. 1 is a graph showing that immunization with the Peptide R16 (SEQ ID No:1) protects retinal ganglion cells (RGCs) from glutamate toxicity in Lewis rats. RGCs of adult Lewis rats were exposed directly to glutamate toxicity by intravitreal injection of L-glutamate (400 nmol). Immediately thereafter, the rats were immunized with 30 μg of R16 emulsified in CFA (0.5 mg/ml). Control rats were injected with PBS in CFA. Two weeks later the optic nerves were exposed for the second time, and the fluorescent dye 4-Di-10-Asp was applied distally to the injury site. Five days after dye application the retinas were detached from the eyes and prepared as flattened whole mounts. Labeled RGCs from four randomly selected fields of identical size in each retina (all located at approximately the same distance from the optic disk) were counted under the fluorescence microscope, and the percentage of RGC loss was calculated and expressed as mean % ±SEM. The percentage of loss was significantly smaller in the R16-immunized rats than in their matched PBS-injected controls (14±2% and 28±4%, respectively; p<0.04 by two-tailed t test). Each group consisted of five or six rats.

FIG. 2 is a graph showing that immunization of Fisher and SPD rats with R16 immediately after optic nerve injury protects their RGCs from secondary death. Adult Fisher and SPD rats were subjected to partial optic nerve crush injury. Immediately thereafter, the rats were immunized with 30 μg of R16 emulsified in CFA (2.5 mg/ml). Control rats were injected with PBS in CFA. Staining with 4-Di-10-Asp, preparation of retinal slides, and counting of labeled RGCs were as described for FIG. 1. The average number of RGCs per square millimeter was calculated. Significantly more RGCs (mean±SEM per square millimeter) survived in the R16-immunized injured rats than in their matched PBS-injected controls (150±13 and 60±14, respectively (p<0.01, two-tailed t test) for SPD rats; 183±16 and 114±9, respectively (p<0.01, two-tailed t test) for Fisher rats). Each group consisted of five or six rats.

FIGS. 3A-D show that immunization of Lewis rats with R16 immediately after optic nerve injury protects their RGCs from secondary death. Adult Lewis rats were subjected to partial optic nerve crush injury. Immediately thereafter, the rats were immunized with 30 μg R16 emulsified in CFA (2.5 mg/ml). Control rats were injected with PBS in CFA. Staining with 4-Di-10-Asp, preparation of retinal slides, and counting of labeled RGCs were as described for FIG. 1. 3A. The average number of RGCs per square millimeter was calculated. Significantly more RGCs (mean±SEM per square millimeter) survived in the R16-immunized injured rats than in their matched PBS-injected controls (192±8 and 73±10 respectively; p<0.0001, by two-tailed t test). Each group consisted of five or six rats. 3B and 3C are representative fluorescence micrographs of PBS-injected injured Lewis rats (3B) and R16-immunized injured Lewis rats (3C). 3D, Survival of RGCs in Lewis rats after optic nerve injury and passive transfer of splenocytes from R16-immunized rats. As controls we used Lewis rats injected with PBS or naïve splenocytes after optic nerve injury. Each group consisted or five or six rats.

FIG. 4 is a graph showing that immunization of Fisher rats (but not Lewis rats) with R16 one week before optic nerve injury protects their RGCs from secondary death. Adult Fisher and Lewis rats were immunized with 30 μg of R16 emulsified in CFA (2.5 mg/ml). Control rats were injected with PBS in CFA. One week later, the rats were subjected to partial optic nerve crush injury and, immediately thereafter, were given a booster injection of 30 μg of R16 emulsified in IFA. Control rats were injected with PBS in IFA. Staining with 4-Di-10-Asp, preparation of retinal slides, and counting of labeled RGCs were as described for FIG. 1. The average number of RGCs per square millimeter was calculated. Significantly more RGCs (mean±SEM per square millimeter) survived in the R16-immunized injured Fisher rats than in their matched PBS-injected controls (165±22 and 89±10, respectively; p<0.01, by two-tailed t test). The difference observed between the R16-immunized and PBS-injected Lewis rats (117±21 and 95±22, respectively) was not statistically significant. Each group consisted of five or six rats.

FIGS. 5A-5B are graphs showing that immunization of Fisher rats with the peptides G-8 (SEQ ID No:4), G-8 analog (SEQ ID No:5), M-8 (SEQ ID No:6), or M-8 analog (SEQ ID No:7), immediately after optic nerve injury, protects their RGCs from secondary degeneration Adult Fisher rats were subjected to partial optic nerve crush injury and then immunized with peptides emulsified in CFA (2.5 mg/ml). Control injured rats were injected with PBS in CFA. Staining with 4-Di-10-Asp, preparation of retinal slides, and counting of labeled RGCs were as described for FIG. 1. 5A, The average number of RGCs per square millimeter was calculated. Significantly more RGCs (mean±SEM per square millimeter) survived in injured rats immunized with 200 μg of G-8, M-8, or M-8 analog than in their matched PBS-injected controls (p<0.01, p<0.03, and p<0.04, respectively, by two-tailed t test). 5B, Significantly more RGCs survived in injured rats immunized with 500 μg of G-8 analog than in their matched PBS-injected controls (p<0.01, by two-tailed t test). Each group consisted of five or six rats.

FIG. 6 is a graph showing that immunization with R16 has no effect on recovery after spinal cord contusion. Female Lewis rats were subjected to spinal contusion at T8. Immediately after contusion, rats in one group (n=5) were immunized with R16 emulsified in CFA, and rats in the other group (n=4) were injected with PBS emulsified in CFA. The motor behavior of each rat was assessed weekly in an open field by observers blinded to the treatment received by the rat. Immunization with R16 did not affect spinal cord recovery. Results are mean values of the motor score ±SEM.

### DETAILED DESCRIPTION OF THE INVENTION

The present invention provides a method for treating a disease, disorder or injury in an organ which is susceptible to a T-cell-mediated specific autoimmune disease, wherein said organ disease, disorder or injury is other than an autoimmune disease, the method comprising immunizing an individual having such a disease, disorder or injury with an agent selected from the group consisting of a pathogenic self-antigen associated with a T-cell-mediated specific autoimmune disease of said organ, a peptide which sequence appears in the sequence of said pathogenic antigen, and an analog of said peptide obtained by replacement of one or more amino acid residues of the original peptide such that the modified peptide is still capable of recognizing the T-cell receptor recognized by the parent peptide but with less affinity. The agent according to the invention may also be a nucleotide sequence encoding the pathogenic self-antigen, the peptide thereof or the analog thereof. The agent may further be T cells activated by said antigen, peptide or peptide analog.

Any tissue-specific self-pathogen may be used as the protective self-antigen according to the invention. Thus, the pathogenic antigen may be associated with the pancreas and the antigen itself, a peptide thereof or an analog of said peptide will be used to treat a non-autoimmune disease of the pancreas, and similarly with respect to any other organ which is susceptible to a T-cell mediated autoimmune disease.

In one preferred embodiment of the invention, the organ is the eye and the pathogenic self-antigen is associated with a T-cell-mediated eye-specific autoimmune disease. In a more preferred embodiment, the pathogenic self-antigen is an uveitogenic antigen associated with autoimmune uveitis, a T cell-mediated autoimmune disease of the eye, and said uveitogenic antigen may be selected from, without being limited to, interphotoreceptor retinoid-binding protein (IRBP), S-antigen (S—Ag) and rhodopsin.

In one preferred embodiment, the antigen is IRBP, a glycolipoprotein with a four-fold partially homologous repeat structure approximately 300 residues in length, one of the retinal antigens capable of inducing EAU in susceptible animals by their immunization (Inoue et al, 1994), and it may be the human, bovine or monkey IRBP.

The present invention encompasses also uveitogenic peptides derived from the IRBP sequence which are capable to cause proliferation of lymphocytes isolated from a significant number of patients suffering from various eye diseases of autoimmune etiology such as Behcet's disease, birdshot retinochoroidopathy, pars planitis, ocular sarcoid, sympathetic ophthalmia, and the Vogt-Koyanagi-Harada syndrome. IRBP and the uveitogenic peptides derived from the IRBP sequence have been described for the treatment of autoimmune uveoretinitis in U.S. Pat. No. 5,961,977, hereby incorporated by reference as if fully disclosed herein.

Thus, in one preferred embodiment of the method of the invention, the organ is the eye and the agent for treatment of a non-autoimmune disease of the eye is selected from the group consisting of:
(a) interphotoreceptor retinoid-binding protein (IRBP);
(b) a peptide which sequence is comprised within the sequence of IRBP;
(c) a peptide obtained by modification of the peptide of (b), which modification consists in the replacement of one or more amino acid residues of the peptide by different amino acid residues, said modified peptide still being capable of recognizing the T-cell receptor recognized by the parent peptide but with less affinity (hereinafter “modified peptide”);
(d) a nucleotide sequence encoding IRPB, a peptide of (b), or a modified peptide of (c); and
(e) T cells activated by an agent selected from the group consisting of IRPB a peptide of (b), and a modified peptide of (c).

Among the peptides derived from the IRBP sequence, preferred peptides according to the invention are the peptides of SEQ ID NO:1, also known as Peptide R16, an immunodominant sequence within IRBP known to cause uveitis (Inoue et al, 1994;), and the peptides of SEQ ID NO:2 and SEQ ID NO:3, also known as Peptides R14 and R4, respectively, all disclosed in the above-mentioned U.S. Pat. No. 5,961,977. The sequences of the peptides R16, R14 and R4 correspond to the amino acid sequences 1177-1191, 1169-1191, and 1158-1180, respectively, from the bovine IRBP.

Thus, the invention comprises the use of a peptide which sequence is comprised within the sequence of IRBP, wherein said peptide is selected from the group consisting of the peptides:

| | |
|---|---|
| ADGSSWEGVGVVPVD; | (SEQ ID NO:1) |
| PTARSVGAADGSSWEGVGVVPDV; | (SEQ ID NO:2) |
| and | |
| HVDDTDLYLTIPTARSVGAADGS. | (SEQ ID NO:3) |

In another embodiment of the invention, the pathogenic ocular autoantigen is the retinal uveitogenic antigen S—Ag, a soluble photoreceptor cell protein having an apparent molecular weight of about 48 kDa, that has been found in all mammalian eyes to date, but bovine eyes are the preferred source because of ready accessibility and similarity to the human S—Ag. The sequence of the human S—Ag is disclosed in U.S. Pat. No. 5,961,977. The complete amino acid sequences of bovine, human and mouse S—Ag have been published elsewhere (Shinohara et al., 1986).

The present invention contemplates the use both of the S—Ag and of fragments derived from the S—Ag sequence as disclosed in U.S. Pat. No. 5,961,977, hereby incorporated by reference. S—Ag and the peptides derived from the S—Ag sequence have been described for the treatment of autoimmune uveoretinitis in U.S. Pat. No. 5,961,977, hereby incorporated by reference as if fully disclosed herein.

Thus, in another preferred embodiment of the method of the invention, the organ is the eye and the agent for treatment of a non-autoimmune disease of the eye is selected from the group consisting of:
(a) S-antigen (S—Ag);
(b) a peptide which sequence is comprised within the sequence of S—Ag;
(c) a peptide obtained by modification of the peptide of (b), which modification consists in the replacement of one or more amino acid residues of the peptide by different amino acid residues, said modified peptide still being capable of recognizing the T-cell receptor recognized by the parent peptide but with less affinity (hereinafter “modified peptide”);
(d) a nucleotide sequence encoding S—Ag, a peptide of (b), or a modified peptide of (c); and
(e) T cells activated by an agent selected from the group consisting of S—Ag, a peptide of (b), and a modified peptide of (c).

In a preferred embodiment, the invention comprises the use of a peptide which sequence is comprised within the sequence of S—Ag, wherein said peptide is selected from the group consisting of the peptides:

| | |
|---|---|
| TSSEVATE; | (SEQ ID NO:4) |
| DTNLASST; | (SEQ ID NO:6) |
| DTNLASSTIIKEGIDKTV; | (SEQ ID NO:8) |
| VPLLANNRERRGIALDGKIKHE; | (SEQ ID NO:9) |
| TSSEVATEVPFRLMHPQPED; | (SEQ ID NO:10) |
| SLTKTLTLVPLLANNRERRG; | (SEQ ID NO:11) |
| SLTRTLTLLPLLANNRERAG; | (SEQ ID NO:12) |
| KEGIDKTVMGILVSYQIKVKL; | (SEQ ID NO:13) |
| and | |
| KEGIDRTVLGILVSYQIKVKL. | (SEQ ID NO:14) |

In another preferred embodiment, the invention comprises the use of an analog of a peptide which sequence is comprised within the sequence of S—Ag, wherein said peptide is selected from the group consisting of the peptides:

| | |
|---|---|
| TSSEAATE; | (SEQ ID NO:5) |
| and | |
| DTALASST. | (SEQ ID NO:7) |

The peptide of SEQ ID NO:4, herein designated Peptide G-8, corresponds to the sequence 347-354 of human retinal soluble Ag (S—Ag), and the peptide of SEQ ID NO:5 is a G-8 analog, in which the valine (V) residue at position 351 was replaced by alanine (A). The peptide of SEQ ID NO:6, herein designated Peptide M-8, corresponds to the sequence 307-314 of human retinal S—Ag, and the peptide of SEQ ID NO:7 is an M-8 analog, in which the asparagine (N) residue at position 309 was replaced by alanine (A). G-8 and M-8 are uveitogenic, while their analogs are immunogenic, but not immunopathogenic (Singh et al, 1994).

Thus, the most preferred embodiment of this invention consists in the use of analogs of the peptides derived from the pathogenic antigen that are immunogenic, but not immunopathogenic.

EAU is an experimental model for uveitis, a T cell-mediated autoimmune disease of the eye. (Prendergast et al, 1998). In the examples hereinafter, we have tested our working hypothesis (namely, that the protective and the destructive autoimmune response share the same antigenic specificity) by investigating whether a pathogenic, uveitis-related retinal self-antigen can protect against direct and indirect insults to the RGCs. The results showed that RGCs exposed to a glutamate insult or suffering the secondary consequences of an optic nerve crush injury could be protected by vaccination with a uveitis-associated peptide, under conditions where no such protective effect could be obtained by vaccination with myelin antigens such as MBP.

According to the present invention, it is shown that self-antigen associated with uveitis protects the retinal ganglion cells from death induced by glutamate or as a consequence of axonal injury. Specifically, vaccination with the peptide R16 (SEQ ID NO:1), an IRBP-derived peptide, resulted in post-injury protection of RGCs, under conditions where no such protective effect could be obtained by vaccination with myelin antigens such as MBP. It is suggested that protective autoimmunity is the way in which the body's defense mechanism against self-destructive compounds is manifested. It is further suggested that an autoimmune disease is a manifestation of an antigen-specific response that was not properly controlled. Thus, the antigenic specificity of a protective autoimmune response can be inferred from the specificity of the autoimmune disease associated with the same tissue, irrespective of the type of insult.

The results according to the invention show that an immunodominant self-antigen causing an autoimmune disease of the eye, EAU, is the same antigen as that inducing protection of RGCs after either mechanical or biochemical insult to the retina or the optic nerve. Until very recently, autoimmunity was defined as a destructive attack of the immune system against a tissue(s) of the body. Several observations, however, are apparently inconsistent with this concept. For example, a high incidence of autoimmune T cells is found in healthy individuals, and disease severity is found not to be correlated with the number of autoimmune T cells.

The results herein show that the self-antigen associated with uveitis protects RGCs from both glutamate toxicity and death induced as a consequence of axonal injury. This protective potential is not restricted to the R16 peptide, as two uveitogenic peptides derived from another retinal antigen, S—Ag, the peptides G-8 and M-8, exerted a similar protective effect in the rat optic nerve injury model. In addition, analogs of the peptides G-8 and M-8, designed to evoke an immune response without causing disease, enhanced RGC survival after optic nerve injury, suggesting that retinal antigens can be used to protect RGCs without the risk of developing autoimmune disease. It is important to emphasize that the protection is antigen-specific, as the protection of RGCs from death caused by a direct insult (such as glutamate toxicity) is conferred by vaccination with peptide R16, but not with myelin antigens, while the opposite is true for injury to the spinal cord. In the case of injury to the optic nerve, however, vaccination with either myelin antigens (Kipnis et al, 2002; Fisher et al, 2001) or retinal antigens improved RGC survival, presumably by attenuating secondary degeneration at the injury site or in the retina, respectively (Schwartz et al, 1999).

An interesting finding of the present invention was that EAU, an autoimmune disease that affects both the anterior and posterior parts of the eye, can cause loss of RGCs. This loss, however, is minor when weighed against the potential benefit of the autoimmune response. Loss of RGCs recorded when the disease resolved itself showed that the maximal loss measured 2 weeks after vaccination in non-injured Lewis rats was ˜17%, whereas the maximal benefit after a neuronal insult was as high as 263% (192±8 surviving RGCs/mm² in rats immunized with R16 compared with 73±10 in rats injected with PBS). These results show that even if the autoimmune response to the uveitogenic antigen causes some loss of RGCs, this cost is outweighed by the benefit that the neurons derive under injurious conditions.

In our view, any tissue uses certain safeguard in its front line of self-defense. We suggest that the antigen that operates evokes an immune response that, in the event of malfunction, induces disease, but not necessarily in the cells that conveyed the stress signal. It thus appears that the tissue endangers some cells for the purpose of saving others. The cells at risk by the disease are neither the RGCs in uveitis nor the myelinated CNS neurons in EAE. Nevertheless, in the absence of appropriate regulation, the intensive autoimmune response against myelin antigens in EAE or against IRBP or S—Ag in uveitis, might eventually lead to neuronal loss as well. Thus, it is shown here that an anti-IRBP response in uninjured Lewis rats can indeed lead to some RGC loss.

It is shown herein in the application that immunization with the uveitis-associated R16 antigen protects RGCs in animals from glutamate toxicity and protects RGCs from secondary degeneration after optic nerve crush. For the immunization, any suitable oil-based or alum-based adjuvant may be used. The choice of antigen and adjuvant may determine the efficacy of the evoked neuroprotective response. In order to reduce the risk of pathogenic autoimmunity while retaining the benefit of neuroprotection, immunization can be carried out with peptides whose pathogenic properties have been weakened. Further optimization of non-pathogenic uveitogenic antigen-derived peptides can be expected to lead to the development of an effective immunization protocol as a therapeutic strategy to treat injuries or disorders in the eye.

The present invention further relates to T cells activated by an uveitogenic antigen, or by a peptide therefrom or by a modified peptide as defined herein. The T cells may be semi-allogeneic but are preferably autologous. To derive the maximum to fully benefit from autoimmune neuroprotection, activated anti-self T cells used for immunization should be “safe”, i.e., they should be able to confer the benefit of protection without the accompanying risk of autoimmune disease. It is important to emphasize that unlike therapies for autoimmune disease, which are based on immune deviation, or tolerance, or response even from general immunosuppression, immune neuroprotective therapy is based on active T cell anti-self response which is insufficiently effective in its spontaneous form and is therefore in need of boosting. In the case of an injury in the eye, therapy should be administered as soon as possible after the primary injury to maximize the chances of success, preferably within about one week.

The present invention further provides pharmaceutical compositions comprising the antigen as defined herein, a peptide derived from said antigen or an analog of said peptide, and a pharmaceutically acceptable carrier. The carrier(s) must be “acceptable” in the sense of being compatible with the other ingredients of the composition and not deleterious to the recipient thereof. The pharmaceutical compositions are prepared by conventional means as well-known in the art.

Methods of administration include, but are not limited to, parenteral, e.g., intravenous, intraperitoneal, intramuscular, subcutaneous, mucosal (e.g., oral, intranasal, buccal, vaginal, rectal, intraocular), intrathecal, topical and intradermal routes. Administration can be systemic or local. Pharmaceutical compositions comprising an antigen, a peptide or a modified peptide according to the invention may optionally be administered with an adjuvant.

Additionally, the antigen, peptide or modified peptide may be used for in vivo or in vitro activation of T cells. For example, a subject can initially be immunized with the antigen, peptide or modified peptide. A T-cell preparation can be prepared from the blood of such immunized subjects, preferably from T cells selected for their specificity towards the antigen. Following their proliferation in vitro, the T cells are administered to a subject in need. In a preferred embodiment, the T cells are autologous. The activated T cells of the invention can be used immediately or may be preserved for later use, e.g., by cryopreservation as known in the art. Said activated T cells may also be obtained using previously cryopreserved T cells, i.e., after thawing the cells, the T cells may be incubated with the antigen, peptide or modified peptide, optimally together with thymocytes.

In one preferred embodiment, the method of the invention is directed to the treatment of a disease, disorder or injury in the eye, wherein said eye disease, disorder or injury is other than an autoimmune disease.

Any non-autoimmune eye injury may be treated according to the invention such as blunt trauma caused by an agent selected from the group consisting of foreign bodies, contusion, laceration, burns or laser surgery.

In addition, any non-autoimmune eye disorder may be treated according to the invention such as glaucoma or another eye disorder selected from the group consisting of a conjunctival, a corneal, a retinal, and an optic nerve or optic pathway disorder. The conjunctival disorder may be selected, for example, from the group consisting of acute conjunctivitis, viral conjunctivitis, bacterial conjunctivitis, and scleritis. The corneal disorder may be selected, for example, from the group consisting of corneal ulcer, herpes simplex keratitis, and interstitial keratitis. The retinal disorder may be selected, for example, from the group consisting of a disorder causing injury or death of photoreceptor cells; a viral retinopathy selected from CMV retinopathy and HIV retinopathy; a vascular retinopathy selected from the group consisting of hypertensive retinopathy, diabetic retinopathy, central retinal artery occlusion and central retinal vein occlusion; a retinopathy due to trauma or penetrating lesions of the eye; retinal detachment; age-related macular degeneration; and retinitis pigmentosa. The optic nerve or optic pathway disorder may be selected, for example, from the group consisting of papilledema, papillitis, retrobulbar neuritis, optic atrophy and higher optic pathway lesions. Other eye diseases or disorders that can be treated according to the invention include non-autoimmune uveitis (any non-autoimmune inflammation of the uveal tract, i.e. iris, ciliary body, or choroid).

The invention will be illustrated by the following non-limiting Examples.

### EXAMPLES

Materials and Methods

(i) Animals. Adult male Sprague-Dawley (SPD), Fisher (F344) and Lewis rats (8-12-week old), and adult female Lewis rats (16-18-week old) were supplied by the Animal Breeding Center of the Weizmann Institute of Science (Rehovot, Israel) under germ-free conditions. The rats were housed in a light- and temperature-controlled room and were matched for age in each species for each experiment. Animals were handled according to the regulations formulated by the Institutional Animal Care and Use Committee.

(ii) Antigens. The peptides R16 (SEQ ID NO:1), G-8 (SEQ ID NO:4), G-8 analog (SEQ ID NO:5), M-8 (SEQ ID NO:6), and an M-8 analog (SEQ ID NO:7) were prepared in the Synthesis Unit at the Weizmann Institute of Science (Rehovot, Israel).

(iii) Partial crush injury of the rat optic nerve. The optic nerve was subjected to a well-calibrated crush injury, as previously described (Yoles and Schwartz, 1998a). Briefly, rats were deeply anesthetized by intraperitoneal (i.p.) injection of Rompun (xylazine, 10 mg/kg; VMD, Arendonk, Belgium) and Vetalar (ketamine, 50 mg/kg; Fort Dodge Laboratories, Fort Dodge, Iowa). Using a binocular operating microscope, lateral canthotomy was performed in the right eye, and the conjunctiva was incised laterally to the cornea. After separation of the retractor bulbi muscles, the optic nerve was exposed intraorbitally by blunt dissection. Using calibrated cross-action forceps, the optic nerve was subjected to a severe crush injury 1-2 mm from the eye. The contralateral nerve was left undisturbed.

(iv) Glutamate injection. The right eye of the anesthetized rat was punctured with a 27-gauge needle through the conjunctiva and sclera, anterior to the pars plana so that the retina was untouched, and a 10-μl Hamilton syringe (Reno, Nev.) with 30-gauge needle was inserted as far as the vitreal body. Rats were injected with 2 μl (400 nmol) of L-glutamate.

(v) Active immunization. Rats were subjected to optic nerve crush injury and then immediately immunized by s.c. injection at the base of the tail of R16 (30 μg), G-8, G-8 analog, M-8, or M-8 analog (200 or 500 μg) emulsified in CFA supplemented with 2.5 mg/ml Mycobacterium tuberculosis (Difco, Detroit, Mich.) in a total volume of 0.1 ml. Rats in another group were exposed to a glutamate insult (by intravitreal glutamate injection), and then immediately immunized s.c. at the base of the tail with 30 μg of R16 emulsified in CFA supplemented with 2.5 or 0.5 mg/ml of M. tuberculosis in a total volume of 0.1 ml. Control rats were injected with PBS in CFA. In another set of experiments, rats were actively immunized with 30 μg of R16 emulsified in CFA supplemented with 2.5 mg/ml M. tuberculosis one week before the crush injury, and given a booster of 30 μg of R16 emulsified in IFA (Difco) immediately after the injury. Control rats were injected with PBS in CFA and boosted with PBS in IFA.

(vi) Passive immunization. Male Lewis rats were bilaterally injected in the hind footpads with 30 μg of R16 emulsified in CFA supplemented with 2.5 mg/ml of Mycobacterium tuberculosis, in a total volume of 0.1 ml. Seven days after immunization, spleens from immunized and naïve rats were removed and pooled in ice-cold PBS. A single-cell suspension was prepared and the cells (2×10⁶ cells/ml) were cultured with naïve thymocytes (2×10⁶ cells/ml) in the presence of R16 (20 μg/ml) in proliferation medium containing Dulbecco's modified Eagle's medium (DMEM) supplemented with 2 mM glutamine, 2-mercaptoethanol (5×10⁻⁵M), sodium pyruvate (1 mM), non-essential amino acids (1 ml/100 ml), 1% fresh autologous rat serum, 100 U/ml penicillin, and 0.1 mg/ml streptomycin. After incubation for 72 h, the cultures were collected and washed, and the lymphoblasts (1.3×10⁷ cells in 3 ml PBS) or PBS alone (3 ml) were injected i.p. into Lewis rats immediately after optic nerve crush injury.

(vii) Assessment of secondary degeneration in the rat optic nerve by retrograde labeling of retinal ganglion cells. Secondary degeneration of optic nerve axons and their corresponding RGCs was evaluated after application, 2 weeks after injury, of the fluorescent lipophilic 4-Di-10-Asp (Molecular Probes Europe, Leiden, The Netherlands), distal to the lesion site. The right optic nerve was exposed for the second time, again without damaging the retinal blood supply. Complete axotomy was performed 1-2 mm distal to the injury site, and solid crystals (0.2-0.4 mm in diameter) of 4-Di-10-Asp were deposited at the site of the new axotomy. Five days after dye application, the rats were killed. Retinas were detached from the eyes, prepared as flattened whole mounts in 4% paraformaldehyde solution, and examined for labeled RGCs by fluorescence microscopy and confocal microscopy. Since only intact axons can transport the dye back to their cell bodies, application of the dye distal to the lesion site 2 weeks after injury ensures that only axons that survived both primary damage and secondary degeneration will be counted. This approach enables us to differentiate between neurons that are still functional and neurons in which the axons are injured but the cell bodies are still viable.

(viii) Spinal cord confusion. Female Lewis rats were anesthetized by i.p. injection of Rompun and Vetalar, and their spinal cords were exposed by laminectomy at the level of T8. One hour after induction of anesthesia, a 10-g rod was dropped onto the laminectomized cord from a height of 50 mm, using the NYU impactor, a device shown to inflict a well-calibrated contusive injury of the spinal cord (Hauben et al, 2000b; Basso et al, 1996).

(ix) Active immunization. Rats were immunized s.c. on a random basis with 100 μg of R16, or injected with PBS, each emulsified in CFA supplemented with 0.5 mg/ml Mycobacterium tuberculosis, in a total volume of 0.1 ml. Rats were immunized within 1 h after contusion.

(x) Animal care. In contused rats, bladder expression was assisted by massage at least twice a day (particularly during the first 48 h after injury, when it was performed three times a day) throughout the experiment. All rats were carefully monitored for evidence of urinary tract infection or any other sign of systemic disease. During the first week after contusion and in any case of hematuria after that period, they received a course of sulfamethoxazole (400 mg/ml) and trimethoprim (8 mg/ml; Resprim; Teva Pharmaceutical Industries, Ashdod, Israel), administered orally with a tuberculin syringe (0.3 ml solution/day). Daily inspections included examination of the laminectomy site for evidence of infection and assessment of the hind limbs for signs of autophagia or pressure.

(xi) Assessment of recovery from spinal cord contusion. Behavioral recovery was scored in an open field using the locomotor rating scale of Basso, Beattie, and Bresnahan, where a score of 0 registers complete paralysis and a score of 21 indicates complete mobility (Basso et al, 1996). Blind scoring ensured that observers were not aware of the treatment received by individual rats. Approximately once a week, the locomotor activities of the trunk, tail, and hind limbs were evaluated in an open field by placing each rat for 4 min in the center of a circular enclosure (90 cm diameter, 7-cm wall height) made of molded plastic with a smooth, non-slip floor. Before each evaluation, the rats were examined carefully for perineal infection, wounds in the hind limbs, and tail and foot autophagia (Hauben et al, 2001b; Rapalino et al, 1998).

### Example 1

### Uveitogenic Peptide Derived from IRBP Protects Against Glutamate-Induced RGC Loss

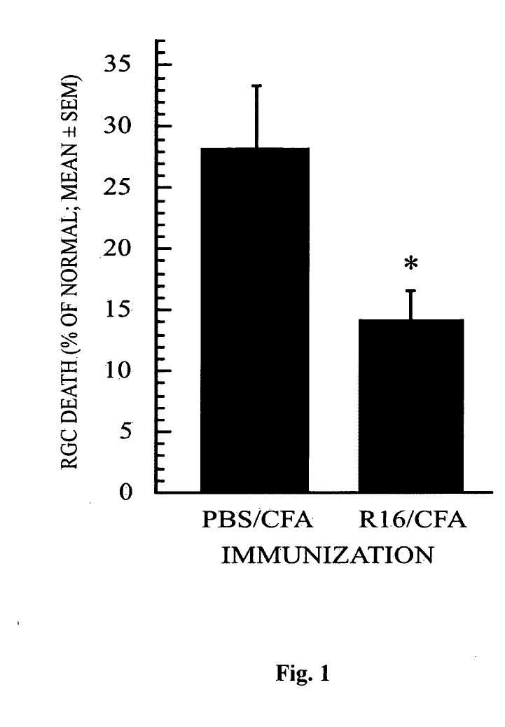

To test our working hypothesis, we first investigated whether RGCs can be protected by vaccination with a self-peptide associated with uveitis, an autoimmune disease affecting the eye. The peptide selected for this experiment was R16 (SEQ ID NO:1), an immunodominant sequence within IRBP known to cause uveitis. First we examined whether vaccination with R16 could protect the RGCs of Lewis rats (a strain susceptible to autoimmune disease induction) from glutamate toxicity under conditions where immunization with myelin peptides was not effective (Schori et al, 2001b). Vaccination of Lewis rats with R16 after a glutamate insult indeed resulted in a reduced loss of RGCs (FIG. 1). Relative to normal retinas, the percentage of RGC loss (mean±SEM) was 14±2% in rats vaccinated with R16 emulsified in CFA compared with 28±4% in rats treated with PBS in CFA (p<0.04). This finding substantiates our contention that immune protection requires the activity of T cells specific to antigen present within the injured tissue. Since R16 is known to cause uveitis in Lewis rats (but not in SPD or Fisher rats), it was interesting to discover that the RGCs received protection despite massive infiltration of lymphocytes into the eyes of these rats. It is important to mention, however, that the neuroprotective effect of R16 in this model was detected only when the disease in these rats was mild (i.e. when the amount of bacteria in the adjuvant was 0.5 mg/ml). Immunization of glutamate-injected rats with R16 emulsified in CFA at a concentration of 2.5 mg/ml was not protective and even caused additional neuronal loss compared with that seen in rats immunized with PBS in CFA (data not shown). These findings constitute further evidence of the delicate balance between the processes of destruction and protection attributable to these specific autoimmune T cells.

### Example 2

### Uveitogenic Peptide Derived from IRBP Protects Retinal Ganglion Cells from the Consequences of Optic Nerve Injury

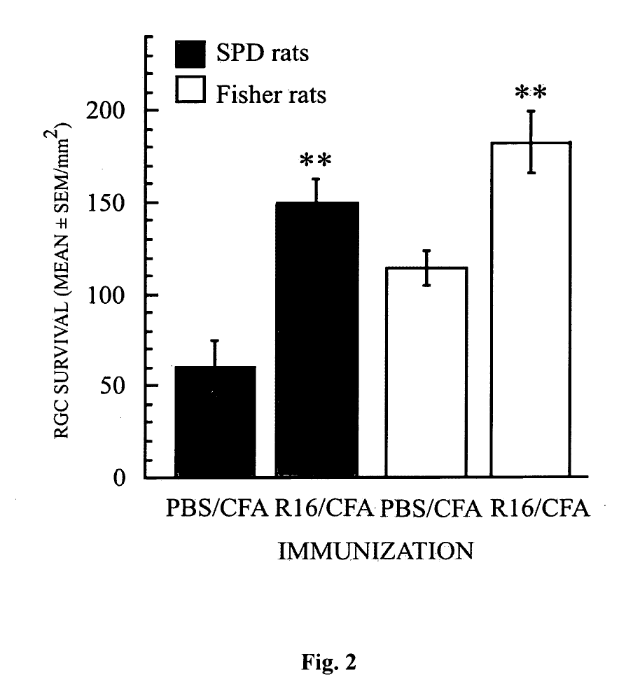

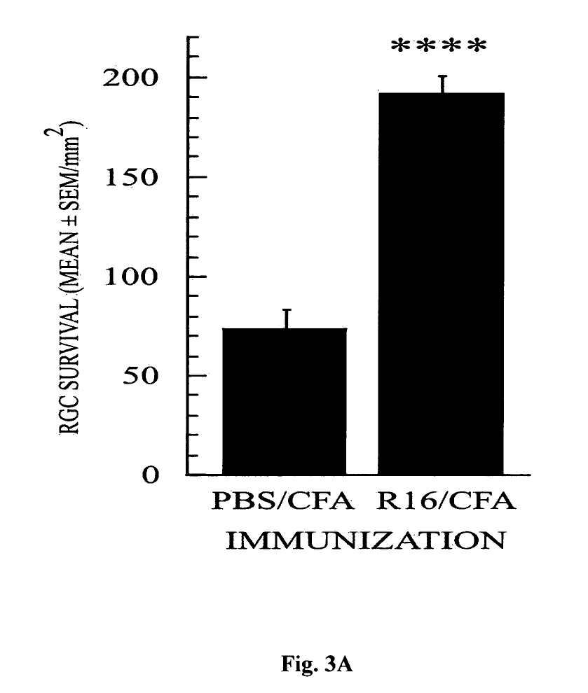

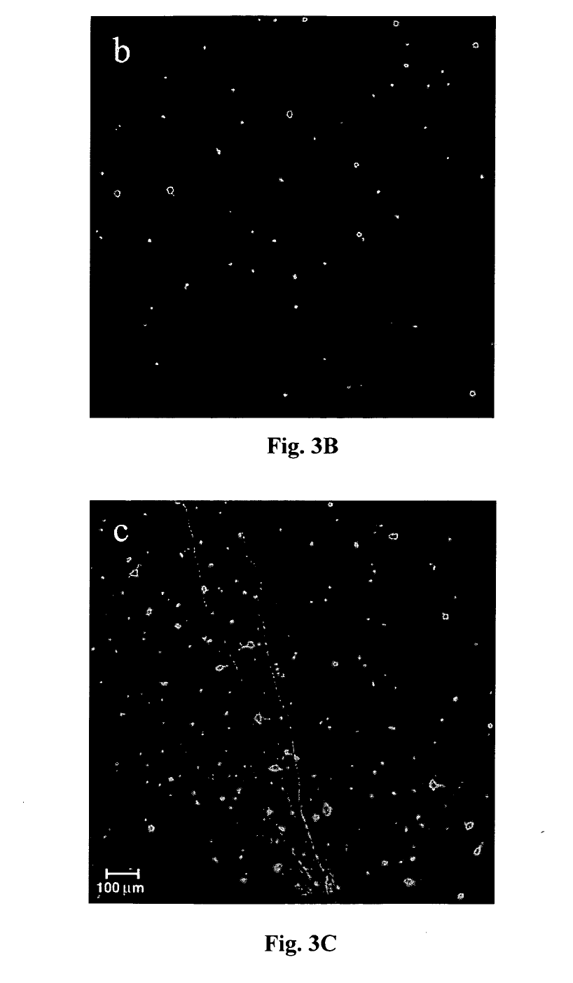

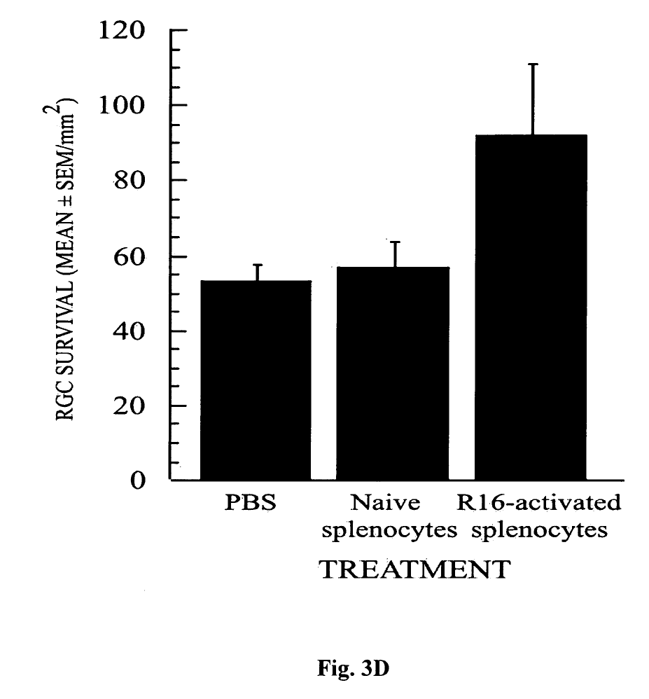

Next we examined the effectiveness of R16 vaccination in protecting RGCs from secondary degeneration after optic nerve crush, an insult known to trigger secondary degeneration initiated in the cell bodies or axons of neurons that escaped direct injury (Yoles and Schwartz, 1998b). This examination was conducted in the two resistant rat strains (SPD and Fisher) and in the susceptible strain (Lewis). In all three strains, vaccination with R16 emulsified in CFA (with the high bacterial content of 2.5 mg/ml) on the day of injury significantly reduced the injury-induced loss of RGCs (FIGS. 2 and 3). In SPD rats, the number of surviving RGCs per square millimeter (mean±SEM) was 150±13 in rats immunized with R16 in CFA and 60±14 in rats injected with PBS in CFA (p<0.01; FIG. 2). The corresponding results were 183±16 and 114±9, respectively, in Fisher rats (p<0.01; FIG. 2), and 192±8 and 73±10, respectively, in Lewis rats (p<0.0001; FIG. 3, A-C). Based on the antigenic specificity found in the case of glutamate toxicity, we attributed the dramatic protection of RGCs observed after R16 vaccination in the crush model to protection by T cells that had migrated to the retina and become activated there, rather than to protection adjacent to the lesion site.

To verify that the observed protection is mediated by T cells, we transferred R16-activated splenocytes to optic nerve-injured Lewis rats. Passive transfer of splenocytes from R16-immunized Lewis rats to nonimmunized Lewis rats immediately after optic nerve injury resulted in a higher number of surviving RGCs per square millimeter (mean±SEM) in the recipient rats (92±19 compared with 53±4 in PBS-injected rats and 57±6 in rats injected with naïve splenocytes; p<0.03 for the comparison of recipient rats with pooled controls, by ANOVA; FIG. 3D).

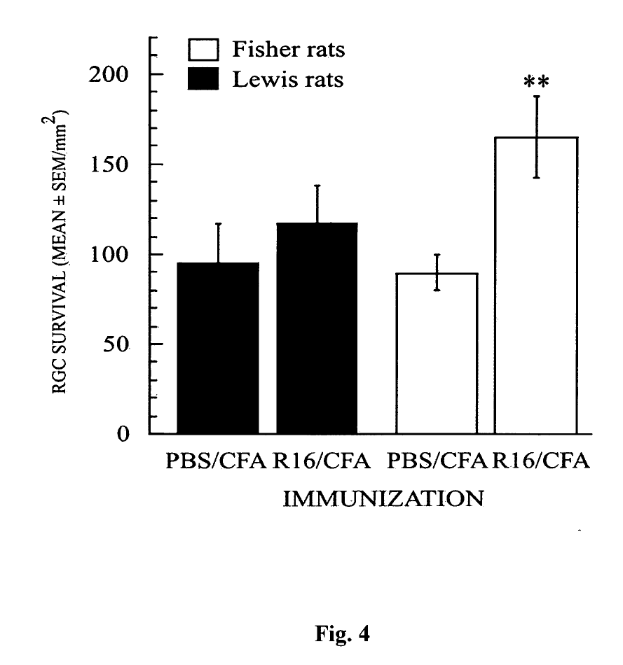

It is interesting to note that the clinical onset of EAU in Lewis rats occurred on day 10 after immunization, and inflammation peaked on day 14. Thus, at the time of assessment of neuronal survival in the crush injury model, the EAU disease in Lewis rats was still severe. We were therefore interested in knowing whether, under such conditions, immunization by itself would have an adverse effect on RGC survival in Lewis rats despite the overall protection. We therefore examined whether R16 immunization in the absence of insult causes any RGC loss in Lewis rats. The percentage of RGC survival (mean±SEM) was significantly lower in the R16-immunized Lewis rats than in their matched PBS-injected controls (83±5%; p=0.02, by one-tailed t test). Immunization with R16 did not affect RGC survival in Fisher rats (97±8% survival). Thus, some loss of RGCs was evident 2 weeks after R16 vaccination in Lewis, but not in Fisher rats, suggesting that uncontrolled autoimmunity leading to autoimmune disease can indeed be destructive in a susceptible strain, but that even in this strain the beneficial effect of autoimmunity on neuronal survival exceeds its destructive effect, so that the net outcome is favorable. In resistant rats, controlled autoimmunity allows the beneficial effect of autoimmunity to be expressed under a wider range of conditions. Support for this suggestion comes from the finding that in the resistant Fisher rats, unlike in the susceptible Lewis rats, vaccination 1 week before injury resulted in significant protection (FIG. 4).

### Example 3

### Peptides Derived from S—Ag Protect Against Retinal Ganglion Cell Loss as a Consequence of Optic Nerve Injury

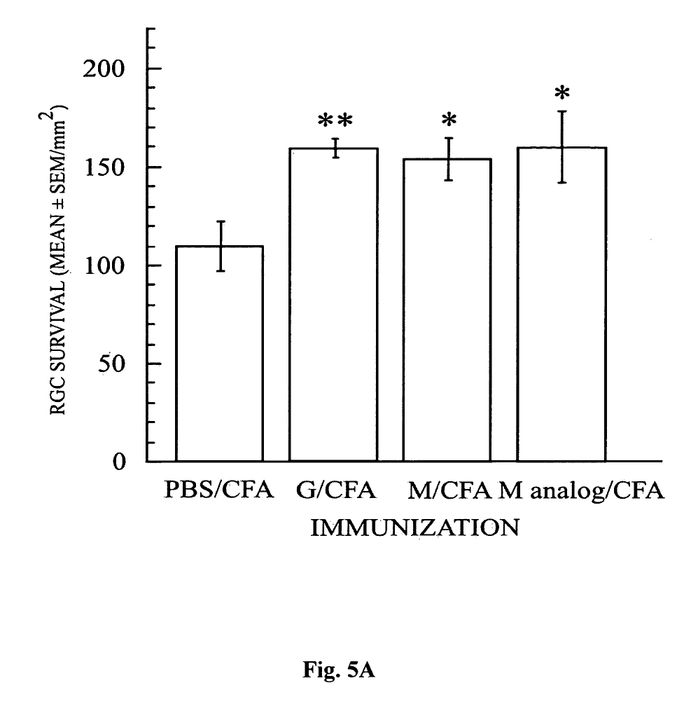

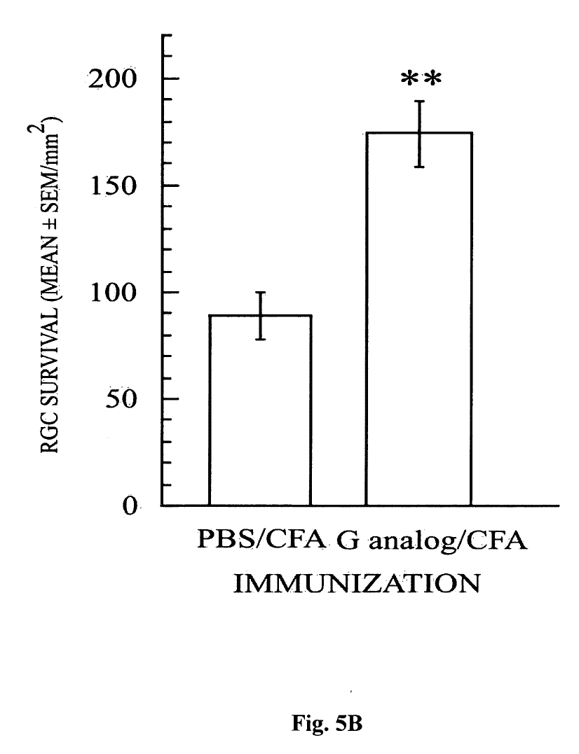

To gain further support for the idea that the protective response is antigen-specific, we used two additional uveitogenic epitopes, G-8 (SEQ ID NO:4) and M-8 (SEQ ID NO:6) of another retinal autoantigen, S—Ag, and their immunogenic, but not immunopathogenic, analogs (SEQ ID NO:5. and SEQ ID NO:7, respectively). As with R16, vaccination with the uveitogenic peptides G-8 and M-8 or their immunogenic analogs immediately after optic nerve crush injury, resulted in a significant increase in RGC survival in Fisher rats. The numbers of surviving RGCs per square millimeter (mean±SEM) were 159±5, 153±10, and 159±19 in rats immunized with 200 μg G-8, M-8, or M-8 analog in CFA and 109±12 in rats injected with PBS in CPA p<0.01, p<0.03, and p<0.04, respectively; FIG. 5A). In the case of the G-8 analog, immunization with 500 μg (but not with 200 μg) of the peptide resulted in a significant increase in RGC survival compared with that in rats injected with PBS in CFA (175±15 and 90±11, respectively; p<0.01; FIG. 5B).

### Example 4

### Protection with Uveitogenic Peptide is Restricted to Insults Residing in the Eye

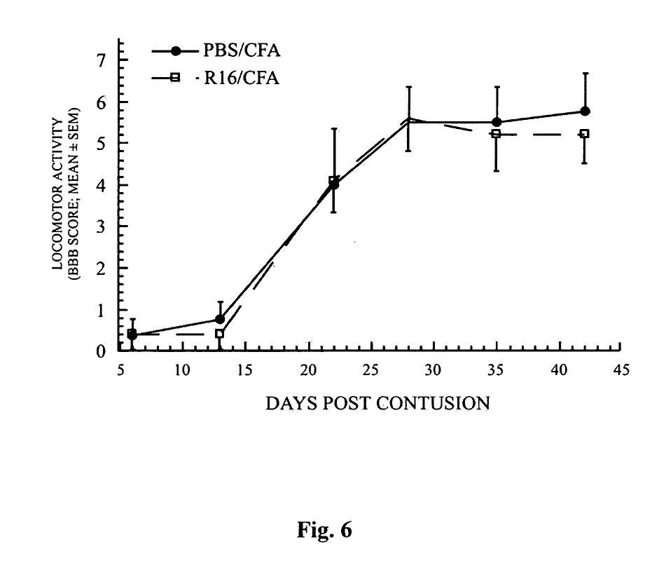

The above results suggest that when a neuronal insult affects the retinal cell bodies directly, immune neuroprotection is restricted to antigens expressed within the retina. This suggests that vaccination with R16 should not protect against injury to the spinal cord, for example, even though spinal cord tissue can benefit from autoimmunity directed to myelin antigens. To examine whether R16 can protect against incomplete spinal cord injury, we subjected Lewis rats to severe spinal cord contusion and then either vaccinated them with R16 in CFA or injected them with PBS in CFA. Recovery was assessed by experimenters who were blinded to the treatment received. At no time were any differences observed in the recovery of motor activity by the two groups (FIG. 6). Under the same experimental conditions in this model, vaccination with a pathogenic peptide derived from myelin basic protein led to better recovery than that seen in non-vaccinated rats (Hauben et al, 2000b; Hauben et al, 2001 a,b).

### REFERENCES

Basso, D. M., M. S. Beattie, and J. C. Bresnahan. 1996. Graded histological and locomotor outcomes after spinal cord contusion using the NYU weight-drop device versus transection. Exp Neurol 139:244.
Fisher, J., H. Levkovitch-Verbin, H. Schori, E. Yoles, O. Butovsky, J. F. Kaye, k Ben-Nun, and M. Schwartz. 2001. Vaccination for neuroprotection in the mouse optic nerve: implications for optic neuropathies. J Neurosci 21:136.
Hauben, E., U. Nevo, E. Yoles, G. Moalem, E. Agranov, F. Mor, S. Akselrod, M. Neeman, I. R. Cohen, and M. Schwartz. 2000. Autoimmune T cells as potential neuroprotective therapy for spinal cord injury [letter]. Lancet 355:286.
Hauben, E., O. Butovsky, U. Nevo, E. Yoles, G. Moalem, E. Agranov, F. Mor, R. Leibowitz-Amit, E. Pevsner, S. Akselrod, M. Neeman, I. R. Cohen, and M. Schwartz. 2000b. Passive or active immunization with myelin basic protein promotes recovery from spinal cord contusion. J Neurosci 20:6421.
Hauben, E., A. Ibarra, T. Mizrahi, R. Barouch, E. Agranov, and M. Schwartz. 2001a. Vaccination with a Nogo-A-derived peptide after incomplete spinal-cord injury promotes recovery via a T-cell-mediated neuroprotective response: comparison with other myelin antigens. Proc Natl Acad Sci USA 98:15173.
Hauben, E., E. Agranov, A. Gothilf, U. Nevo, A. Cohen, I. Smirnov, L. Steinman, and M. Schwartz. 2001b. Posttraumatic therapeutic vaccination with modified myelin self-antigen prevents complete paralysis while avoiding autoimmune disease. J Clin Invest 108:591.
Inoue, H., M. Takeuchi, T. Tanaka, M. Usui, S. Ando, and O. Taguchi. 1994. Analysis of the uveitogenic determinant in repeat structure of retinal interphotoreceptor retinoid-binding protein (IRBP). Clin Exp Immunol 97:219.
Kipnis, J., E. Yoles, H. Schori, E. Hauben, I. Shaked, and M. Schwartz. 2001. Neuronal survival after CNS insult is determined by a genetically encoded autoimmune response. J Neurosci 21:4564.
Kipnis, J., T. Mizrahi, E. Yoles, and M. Schwartz. 2002. Myelin-specific Th1 Cells are Necessary for Post-traumatic Protective Autoimmunity. J Neuroimmunol 130:78.
Moalem, G., R. Leibowitz-Amit, E. Yoles, F. Mor, I. R. Cohen, and M. Schwartz. 1999. Autoimmune T cells protect neurons from secondary degeneration after central nervous system axotomy. Nat Med 5:49.
Moalem, G., E. Yoles, R. Leibowitz-Amit, S. Muller-Gilor, F. Mor, I. R. Cohen, and M. Schwartz. 2000. Autoimmune T cells retard the loss of function in injured rat optic nerves. J Neuroimmunol 106:189.
Prendergast, R. A., C. E. Iliff, N. M. Coskuncan, R. R. Caspi, G. Sartani, T. K. Tarrant, G. A. Lutty, and D. S. McLeod. 1998. T cell traffic and the inflammatory response in experimental autoimmune uveoretinitis. Invest Ophthalmol Vis Sci 39:754.
Rapalino, O., O. Lazarov-Spiegler, E. Agranov, G. J. Velan, E. Yoles, M. Fraidakis, A. Solomon, R. Gepstein, A. Katz, M. Belkin, M. Hadani, and M. Schwartz. 1998. Implantation of stimulated homologous macrophages results in partial recovery of paraplegic rats. Nat Med 4:814.
Schori, H., E. Yoles, and M. Schwartz. 2001a. T-cell-based immunity counteracts the potential toxicity of glutamate in the central nervous system. J Neuroimmunol 119:199.
Schori, H., J. Kipnis, E. Yoles, E. WoldeMussie, G. Ruiz, L. A. Wheeler, and M. Schwartz. 2001b. Vaccination for protection of retinal ganglion cells against death from glutamate cytotoxicity and ocular hypertension: implications for glaucoma. Proc Natl Acad Sci USA 98:3398.
Schwartz, M., E. Yoles, and L. A. Levin. 1999. ‘Axogenic’ and ‘somagenic’ neurodegenerative diseases: definitions and therapeutic implications. Mol Med Today 5:470.
Shinohara, T., et al., 1986. S-Antigen: Structure, Function and Experimental Autoimmune Uveitis (EAU), in Progress in Retinal Research, N. Osborne and J. Chader (Eds.), Pergamon Press, pp. 51-66).
Singh, D. P., T. Kikuchi, V. K. Singh, and T. Shinohara. 1994. A single amino acid substitution in core residues of S-antigen prevents experimental autoimmune uveitis. J Immunol 152:4699.
Yoles, E., and M. Schwartz. 1998a. Degeneration of spared axons following partial white matter lesion: implications for optic nerve neuropathies. Exp Neurol 153:1.
Yoles, E., and M. Schwartz. 1998b. Elevation of intraocular glutamate levels in rats with partial lesion of the optic nerve. Arch Ophthalmol 116:906.
Yoles, E., E. Hauben, O. Palgi, E. Agranov, A. Gothilf, A. Cohen, V. Kuchroo, I. R. Cohen, H. Weiner, and M. Schwartz. 2001. Protective autoimmunity is a physiological response to CNS trauma. J Neurosci 21:3740.

1-29. (canceled)

30. A method for treating a disease, disorder or injury in an organ which is susceptible to a T-cell-mediated specific autoimmune disease, wherein said organ disease, disorder or injury is other than an autoimmune disease, the method comprising immunizing an individual having such a disease, disorder or injury with an agent selected from the group consisting of:
(a) a pathogenic self-antigen associated with a T-cell-mediated specific autoimmune disease of said organ;
(b) a peptide which sequence is comprised within the sequence of said pathogenic self-antigen of (a);
(c) a peptide obtained by modification of the peptide of (b), which modification consists in the replacement of one or more amino acid residues of the peptide by different amino acid residues, said modified peptide still being capable of recognizing the T-cell receptor recognized by the parent peptide but with less affinity (hereinafter “modified peptide”);
(d) a nucleotide sequence encoding a pathogenic self-antigen of (a), a peptide of (b), or a modified peptide of (c); and
(e) T cells activated by a pathogenic self-antigen of (a), a peptide of (b), or a modified peptide of (c).

31. The method of claim 30 wherein said pathogenic self-antigen is associated with a T-cell-mediated eye-specific autoimmune disease.

32. The method of claim 31 wherein said pathogenic self-antigen is an uveitogenic antigen associated with autoimmune uveitis.

33. The method of claim 32 wherein said pathogenic uveitogenic antigen is selected from the group consisting of interphotoreceptor retinoid-binding protein (IRBP), S-antigen (S—Ag) and rhodopsin.

34. The method of claim 33 wherein said pathogenic uveitogenic antigen is IRBP and said agent is selected from the group consisting of:
(a) interphotoreceptor retinoid-binding protein (IRBP);
(b) a peptide which sequence is comprised within the sequence of IRBP;
(c) a peptide obtained by modification of the peptide of (b), which modification consists in the replacement of one or more amino acid residues of the peptide by different amino acid residues, said modified peptide still being capable of recognizing the T-cell receptor recognized by the parent peptide but with less affinity (hereinafter “modified peptide”);
(d) a nucleotide sequence encoding IRPB, a peptide of (b), or a modified peptide of (c); and
(e) T cells activated by an agent selected from the group consisting of IRPB, a peptide of (b), and a modified peptide of (c).

35. The method of claim 34 wherein said peptide (b) which sequence is comprised within the sequence of IRBP is selected from the group consisting of the peptides:

| | |
|---|---|
| ADGSSWEGVGVVPDV; | (SEQ ID NO:1) |
| PTARSVGAADGSSWEGVGVVPDV; | (SEQ ID NO:2) |
| and | |
| HVDDTDLYLTIPTARSVGAADGS. | (SEQ ID NO:3) |

36. The method of claim 33 wherein said pathogenic uveitogenic antigen is S-Antigen and said agent is selected from the group consisting of:
(a) S-antigen (S—Ag);
(b) a peptide which sequence is comprised within the sequence of S—Ag;
(c) a peptide obtained by modification of the peptide of (b), which modification consists in the replacement of one or more amino acid residues of the peptide by different amino acid residues, said modified peptide still being capable of recognizing the T-cell receptor recognized by the parent peptide but with less affinity (hereinafter “modified peptide”);
(d) a nucleotide sequence encoding S—Ag, a peptide of (b), or a modified peptide of (c); and
(e) T cells activated by an agent selected from the group consisting of S—Ag, a peptide of (b), and a modified peptide of (c).

37. The method of claim 36 wherein said peptide (b) which sequence is comprised within the sequence of S—Ag is selected from the group consisting of the peptides:

| | |
|---|---|
| TSSEVATE; | (SEQ ID NO:4) |
| DTNLASST; | (SEQ ID NO:6) |
| DTNLASSTIIKEGIDKTV; | (SEQ ID NO:8) |
| VPLLANNRERRGIALDGKIKHE; | (SEQ ID NO:9) |
| TSSEVATEVPFRLMHPQPED; | (SEQ ID NO:10) |
| SLTKTLTLVPLLANNRERRG; | (SEQ ID NO:11) |
| SLTRTLTLLPLLANNRERAG; | (SEQ ID NO:12) |
| KEGIDKTVMGILVSYQIKVKL; | (SEQ ID NO:13) |
| and | |
| KEGIDRTVLGILVSYQIKVKL. | (SEQ ID NO:14) |

38. The method of claim 36 wherein said modified peptide (c) is selected from the group consisting of the peptides:

| | |
|---|---|
| TSSEAATE; | (SEQ ID NO:5) |
| and | |
| DTALASST. | (SEQ ID NO:7) |

39. The method of claim 31 for treating a disease, disorder or injury in the eye, wherein said eye disease, disorder or injury is other than an autoimmune disease.

40. The method of claim 39 wherein said non-autoimmune eye injury is blunt trauma caused by an agent selected from the group consisting of foreign bodies, contusion, laceration, burns or laser surgery.

41. The method of claim 39 wherein said non-autoimmune eye disorder is selected from the group consisting of a conjunctival, a corneal, a retinal, and an optic nerve or optic pathway disorder.

42. The method of claim 39 wherein said non-autoimmune disorder is glaucoma.
